(19) United States
(12) Patent Application Publication
(10) Pub. No.: US 2022/0291214 A1
(43) Pub. Date: Sep. 15, 2022

(54) Apparatus and Method for Testing the Health Condition of Animals and/or Human Beings

(71) Applicant: Iomics Limited, Cardiff South Glamorgan (GB)

(72) Inventor: David Rooke, Cardiff South Glamorgan (GB)

(21) Appl. No.: 17/628,749

(22) PCT Filed: Aug. 12, 2020
(86) PCT No.: PCT/GB2020/051916

§ 371 Date: Jan. 20, 2022

(30) Foreign Application Priority Data
Aug. 14, 2019 (GB) 1911642.5

(51) Int. Cl. G01N 33/569; G01N 33/543; G16H 10/40

(52) U.S. Cl. CPC G01N 33/5695; G01N 33/54388; G16H 10/40

(57) ABSTRACT

An apparatus and a method for undertaking a test on a person or animal on site. The apparatus includes a body having a portion for contact with a sample obtained from a person/or animal and analysis means, typically one or more biomarkers, to allow the test to be analyzed. Indication means can be provided on the apparatus to illustrate the test result on site and/or the test results can be captured and transmitted to a remote location for subsequent analysis. If deemed required after the analysis, one or more actions to be taken can be recommended to a person who has been tested and/or the person performing the test while at the location of the test and thereby allow rapid testing and actions to be performed if required.

The invention to which this application relates is to the provision of apparatus and a method of testing an animal and/or human condition and, more specifically, but not necessarily exclusively, testing using a sample, such as fluid, from the animal and/or human and, as a result of the testing, to be able to identify specific disease and/or health issues and identify one or more actions which are required to be taken.

Potential diseases and health problems which may occur, are widespread and varied. In order to be able to provide an opportunity to mitigate the problems and/or cure the same, it is important to be able to identify, as accurately as possible, the type of problem and, as a result of that identification, provide information and/or solutions to allow the problem to be addressed.

For example, with regard to animals, problems can occur in the testing of the animals both in terms of being able to gain access to the animals in their environment, perform the test on animals which may not be cooperative and then be able to control the test results in such a way that they can be provided in a condition which is fit for further assessment and identification of the health problem. There is also a need to be able to undertake these tests on as many animals in a particular group or herd of animals as possible so as to provide a larger database and be able to identify widescale health problems and take effective measures to address these problems on a group or herd basis.

There is also a need to be able to identify the problem quickly and thereby address the problem and take necessary steps to solve the same in order to prevent, for example, the spreading of a contagious health problem to other animals in the herd or to other groups of animals.

A further problem which is identified is that with certain health problems, there can be a number of variations of the same and, in certain instances, while a particular treatment may be suitable for one variation of a health problem, the same treatment may not be suitable for use in treatment of other variations of the same health problem. As a result, funding decisions for the payment for the particular types of medication, may be refused on the basis that the clinical benefit of the use of that particular medication cannot be illustrated over a sufficiently large scale. This can mean that medication, which is useful for particular patients, is therefore not provided at all.

An example of this is with regard to the health condition Chronic Obstructive Pulmonary Disease (COPD) which is an umbrella term used to describe progressive lung diseases including emphysema, chronic bronchitis, refractory asthma and which, in general, is characterised by increasing breathlessness. In this case, there is firstly a need to be able to diagnose and distinguish COPD from other lung diseases and thereafter, it is known that some COPD patients only respond to specific drugs; such as some responding to bronchodilators, others may respond to anti-inflammatory drugs, and even within these particular classes, some drugs may work for certain patients but not all. This has led to a refusal to find the use of particular drugs unless they are proven to be effective but the problem is that drug companies can struggle to provide clinical effectiveness results as there is currently no means to determine which drugs will work with which patients and therefore it then becomes a case of trial and error requiring long, and expensive, clinician and laboratory whilst drug companies cannot sell their products and therefore may delay the development of other potential versions of the product.

An aim of the present invention is to provide test apparatus and subsequent analysis methods which allow a particular health problem to be identified, the particular version of the health problem to be identified, and allocated to a particular patient to thereby allow a more detailed and personalised treatment procedure to be provided for that patient. A further aim of the invention is to provide the test apparatus in a form which allows the same to be used on site, at the location of the person or animal and for the test results from that apparatus to be passed for subsequent analysis quickly and effectively.

In a first aspect of the invention, there is provided test apparatus for use in testing for the presence of at least one health condition with regard to an animal or person on which the apparatus is used, said apparatus comprising a body including a means for identifying the said person or animal, a portion with which a sample of tissue and/or fluid from the person or animal is brought into contact and wherein the body includes one or more analysis means, the type of which is selected with respect to the particular test which is to be performed and, after the test is performed, an identification is provided on the said body and/or wirelessly transmitted to a remote location to represent the results of the test.

In one embodiment the analysis means allows the analysis of biomarker antibodies and/or antigens.

In one embodiment antigens will be selected to be used for tests in which an antibody response may not be possible.

In one embodiment, the apparatus further includes a means to allow an indication of the result of the test, to be captured, in one embodiment, by photograph or scan, and then communicated to a database to allow the said indication to be assessed and the result confirmed.

In one embodiment, the analysis at the database, will also recommend, if appropriate, a particular action or a range of actions which are to be performed with regard to that particular person or animal or group of persons or animals.

In one embodiment, the said action or range of actions which are required to be performed, if required, are communicated to the person or persons who have undertaken the tests on site so as to be able to be performed with substantially immediate effect.

In one embodiment, the action or range of actions which are required to be performed, may comprise a combination of actions which should be performed as soon as possible and then subsequent actions which can be performed over a time period.

The ability to undertake the tests on site and then receive advice on site generated from analysis of the test results, means that more effective and immediate actions can be taken to control a health problem, if the same is identified with regard to a particular animal or group of animals or person or group of persons.

In one embodiment, the apparatus includes one or more biomarkers in the body of the apparatus in order to identify the presence, or otherwise of wherein the analysis means includes a series of biomarker antibodies in a form so as to identify the presence, or otherwise of any of bovine tuberculosis, lung diseases including COPD and lung cancer, mycrobacterial disease, such as Johne's disease (mycobacteria avium paratubercolosis (MAP)) prostate and/or bladder cancer.

In one embodiment, the contact of the apparatus with the animal or person or samples therefrom, is such as to allow a lateral flow test to be performed on the apparatus.

In one embodiment, in addition or alternatively another relatively quick point of use analytical apparatus is utilised using a different detection and reading mechanism.

In one embodiment the particular detection method and apparatus used is determined with respect to the test which is required to be performed and the on site analysis required to obtain the test results data. As such, for example, other technologies could be used in the event of use of an antibody not being possible e.g. certain virus particles small peptides etc will not illicit an antibody response and so, if the biomarker identified is a key marker in a biomarker array, using a different detection method.

In one embodiment the apparatus allows a range of on site analysis systems to be used. In one embodiment the different systems can be provided in different bodies or in the same body of the apparatus.

In one embodiment, the indication which is generated by the apparatus is of a particular colour, with a first colour equating to a clear test result and a second or number of further colours equating to a particular health problem and/or type of health problem.

In one embodiment, the body will include a number of biomarker antibodies, with the number, type and/or specific configuration of the biomarker antibodies being selected to suit the particular test which is to be performed.

In one embodiment, the biomarkers which are detected, rely on any or any combination of contact with the person or animals' saliva, urine or blood.

In one embodiment, the analysis of the data received from the test apparatus uses “omics” research which is the use of collective technologies to identify the various molecules resulting from the health problem and their relationship to that problem.

In one embodiment, the actions which are identified, are made accessible to a plurality of users, typically in a secure format, in order to allow the relatively rapid dissemination of information, actions and decisions regarding the condition which, in turn, facilitates remote identification and control of the condition.

In a further aspect of the invention, there is provided a method of identifying and providing, if necessary, one of a range of actions with regard to one or more health conditions of a person or animal, said method comprising the steps of, performing a test on a sample obtained from said person or animal to identify results relating to their said health condition; analysing the test results using one or more analysis systems suitable for the test performed and providing an indication of the test result at the location at which the test is performed and/or transmitting data relating to the test results to a remote location for subsequent analysis.

In one embodiment at least initial analysis is performed on site. In one embodiment the analysis utilises biomarker antibodies and/or antigens.

In one embodiment the test utilises a lateral flow system.

In one embodiment the method includes the steps of comparing the data received from the test to a validated database control to determine the status of the tested health condition, reporting the status of the condition to the user and/or tester and uploading the data and the status determined by the test to at least one database in conjunction with a user identification for the test data;

and/or
storing the test and identification data in a secure manner;
and/or
interrogating said data using one or more algorithms to compute a decision process regarding the condition;
and/or
identifying one or more actions which may be required to be performed as a result of the detected condition status and wherein the test is performed using apparatus which identifies one or more biomarkers identified in a bodily fluid from the person or said animal.

Specific embodiments of the invention are now described with reference to the accompanying drawings wherein:

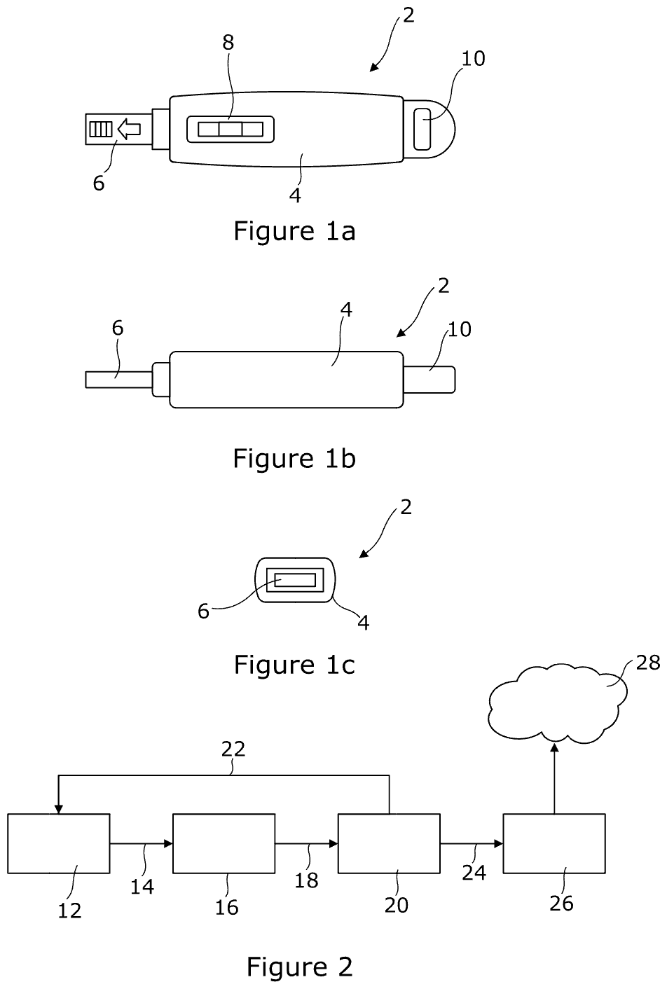

FIGS. 1a-c illustrate a test apparatus in accordance with one embodiment of the invention; and

FIG. 2 illustrates, schematically, a method in accordance with the invention in relation to the testing and analysis of a health condition in accordance with the invention.

Referring firstly to FIGS. 1a-c, there is illustrated apparatus 2 in accordance with one embodiment of the invention which can be provided in a portable form and thereby used at the location of the person or animal which is to be tested. The apparatus comprises a body 4 which includes therein one or more, typically a number, of antibodies which have been selected specifically with regard to a particular health condition which is to be a subject of the test.

The body also includes a portion 6 which can be exposed to a sample of bodily fluid from the person or animal such as blood, serum, extrudates, saliva, sputum, pleural effusion, urine or the like and which leads to the antibodies using lateral flow techniques. The apparatus also means such as a scanner 8 or biomarker signature, also known as biomarker signature, detection means 10 to allow a unique ID to be generated for the patient or animal on which the test is being performed using that item of apparatus 2.

Once the sample has been received on the apparatus as indicated by step 12 in FIG. 2 and exposed to the antibodies the condition of the particular health issue can be identified using biomarker signature technology derived from biomarkers identified, in one embodiment by using “omics” research which is a collective technology used to identify the various molecules resulting from the health condition and the relationship to that health condition using, for example, metabolomics, proteomics, lipidomics, genomics.

The biomarker molecules required in order to perform the particular test for which the apparatus is provided, are identified and then prepared in quantity via current best practice such as via expression, purification, synthesis or the like. These biomarkers then become the antigen component for the development of antibodies such as monoclonal or polyclonal antibodies which are themselves unique to the biomarker.

The antibodies can then be provided in the apparatus 2 in an immobilised condition on a suitable carrier, such as gold nanoparticles but could be another suitable carrier such as magnetic beads or the like. The immobilised particles are incorporated into the disposable point of use diagnostic test apparatus 2 and is based on the principles of immunochromatography and typically a lateral flow test strip.

When the unique biomarker from a sample under test (the analyte) comes into contact with an antibody on the test strip, it gives a visible signal which is measured by the test reading device. This signal identifies the biomarker and the resulting data uploaded to the diagnostic test system.

The number of lanes on the test strip depend upon the number of biomarkers needed for the unique test.

The test data 14 from the apparatus 2 is transmitted from the apparatus 2 via a communication transceiver located in the apparatus body 4 or alternatively via an external apparatus such as a mobile phone to which the apparatus can be connected, wirelessly or via cable connection to a remote database 16 and can then be compared to validated database control parameters to determine the status of the condition with reference to the test ID and the provision of the communication means allows the said data relating to the identifier and/or further data from the particular test apparatus to be uploaded to the database.

The apparatus 2 typically allows the diagnostic test to be based upon the identification of a unique biomarker signature using low-cost, rapid point of view, test mechanisms. A key feature of the biomarker signature is a number of biochemical biomarkers which are associated with the disease and the number of the same can be from one, to tens or more, depending upon the disease and the identifications which need to be made. These are identified via a specialist “omics” research and typically will be a metabolomic biomarker, proteomic biomarker, lipidomic biomarker or the like. It is the ability to identify the signature in addition to the series of biomarkers associated with the health condition to which test relates, which is an important aspect of the invention.

The test result data is then compared to the validated database control at stage 18 to determine the status of the condition and this is a diagnostic stage which allows the detection of the health condition status to be performed for the specific person or animal on which the test was performed.

The status of the condition 20 is identified and the result can be transmitted 22 to the user who may be the patient or carer who typically, will still be on site and can therefore undertake any recommended immediate action, if necessary. Furthermore, the data relating to the health condition can be uploaded 24 along with the status determined by the test, to a clinical and/or other secure database 26 along with a unique user identification such as a tag, number, barcode, QR code, or a biometric identification so as to uniquely associate the diagnostic test with a sample and the user, patients, animal, clinical practitioner, veterinary practitioner and/or the like.

The secure database 26 encrypts and stores the unique test and identification data so that the data can then be interrogated and associated with other metadata using algorithms to compute a decision process in relation to the condition to enable it to be actioned and managed.

By enabling the encrypted data and actions from the decision process algorithm to become accessible on the internet via, for example, cloud hosting 28 to other secure users, the rapid dissemination of information, actions and decisions regarding the condition can be achieved which, in turn, facilitates remote identification and control of the condition.

Typically, the method includes machine learning and artificial intelligence because of the intelligent algorithms which are used. As the data and metadata database increases for any condition, the decision making, which, without this system in a clinical setting, can only be made by qualified practitioners and specialists, can ultimately be taken by the system, hence reducing the burden on the specialist and generation of random errors. This integrated intelligent analysis system in accordance with the invention therefore allows the data to be taken from each test, interprets the result, links it to other data from, for example, a specific animal or patient, and provides, from this, a series of decisions and/or actions which can, if required be relayed immediately to the person on whom the test has been performed and/or the person performing the test.

Management and control elements can be used, for example, in disease management for human and animal diseases, food chain management and farming etc. The current process therefore simplifies the analysis of large amounts of data by using machine learning and powerful artificial intelligence techniques with bioinfomatics. The data is analysed in terms of specific data and patterns of data that are unique to a person and/or animal and can be used to address the manner in which these conditions are currently dealt with and may be in the future.

Furthermore, the method allows the generation of actions and specific treatments which are specific to a person or animal at that time and therefore the treatments can be made more precise to every person and can be monitored subsequently. Patient target therapeutics is therefore possible in accordance with the invention and is performed as a result of tests using low-cost and disposable, rapid test apparatus using lateral flow technology at the point of care of the person or the animal. Due to the fact that the tests can be performed quickly and cheaply, then the same can also be performed more frequently without a significant increase in cost and the use of the secure database information technology, along with intelligent algorithms, allows the data to be used to provide immediate feedback to the clinician, person and/or animal along with the information to decide on the correct course of treatment.

The provision of these person or animal targeted therapeutics enables clinicians to understand and correctly treat the patient, allows drug companies to target their existing products and speedup the clinical evaluation of new drug entities, avoids incorrect and unnecessary prescribing of drugs and allows the health provider to save monies which otherwise would be spent on nontargeted patient care and avoids the environmental impact of overuse of unsuitable drug treatments. Thus, there is provided in accordance with the invention, a diagnostic toolkit, a monitoring system for dynamic changes in the disease and/or the person or animal condition and a guiding system for precision treatment of the health condition.

Specific examples of the use of the invention are provided as follows:

With regard to Chronic Obstructive Pulmonary Disease (COPD) the diagnostic test is carried out at the point of care and is read by the diagnostic test apparatus. The data is captured by the portable test reading device and the results data is transmitted to a remote database, typically using cable or wireless communication means for analysis. After analysis, the patient is advised of the actions required and their carer is notified simultaneously by the system. The risk assessment, decision process and actions are determined and verified with the person, carer, GP, specialist or hospital as required and the data is typically also uploaded to a secure cloud data system for subsequent interrogation and are computed by intelligent algorithms and provide a learning capability which ultimately leads to the completion of the patient's specific personalised companion diagnostic toolkit. This integrated technology is the point of use/care Detect, Manage and Control (DMC) system. For example, with COPD, if a patient is about to have an exacerbation (the specific biomarkers will be apparent before the exacerbation occurs) then by linking the biomarker signature for a specific patient to all the other patient data, a diagnosis and action plan can then be advised to the patient without the need for a consultant intervention. The actions may include for the person to attend hospital immediately, in which case the suitable clinical unit is advised in accordance with the invention to prepare to receive the person. Another action may be to use the biomarker signature and analysis in order to allow the identification, through the data interpretation, patient data and specific pharmacological data, and thereby advise as to the type of treatment actions which should be taken and/or administered.

In another example of the invention, with regard to testing for bovine tuberculosis (bTB), the diagnostic test is carried out at the point of use by a trained veterinary technician/practitioner/farmer because it involves taking a suitable sample from the animal, such as blood. The blood sample is applied immediately to the portion of the body of the apparatus such as a disposable lateral flow test portion. The said portion can then be inserted into analysis means in the form of a diagnostic test device. The animal being tested has a unique identification tag which is read by the diagnostic test reader such as optical character recognition/barcode or QR code and the data from the diagnostic test is captured by the portable apparatus and this is uploaded to a secure database along with the unique ID so that the test data is associated with other data regarding the animal on which test has been performed such as the farm, location etc. Once the diagnostic test and verification of the animal is concluded, the result is notified to the user and can be uploaded to a cloud database for information, notification, action by the farmer and veterinary authorities as appropriate. The data uploaded to the secure cloud data system can be interrogated and computed by intelligent algorithms with its learning capability, along with the track-and-trace via the unique tag can associate the specific animal to all activities and actions along with food chain. For example if the test result shows a presence of tuberculosis the animals and farm linked to the test animal may be sanctioned and this is identified on the general database.

Thus the test method and apparatus has herein defined can have significant and far reaching benefits, for example, continuing with the reference to bTB, with the key biomarkers associated with the disease identified so the next stage is antigen/antibody preparation and then binding these antibodies to the glod particles for the lateral flow test strip of the apparatus.

A number of important features can emerge from the biomarker identification and in this example the biomarker identification is so specific for mycobacterial infection, that by using this one alone one can determine infection with a sensitivity and specificity of >95%—for bTB testing in cattle which is a major step forward and a breakthrough in terms of testing because the current mandated test which uses tuberculin in a skin test not only has a sensitivity of less than 60% (range 40-75% with a lot of variables) but takes 3 days to get a “result” As the “sensitivity” of the test determines the number of false negatives so it is possible with the conventional test that up to half the cows tested “pass” the test but are actually infected and so go back to the herd and as soon as they get stressed/ill (which is very common in high intensity dairy farming) infection increases again.

The government want to introduce a vaccination programme to resolve this (now committed to eradication by 2038) but this is dependent upon a test which can differentiate between vaccinated and infected animals (DIVA)—otherwise vaccination cannot be implemented. In the current invention the identification of the relevant biomarker is important in this respect as it is also DIVA so we have the possibility of a rapid, accurate and highly sensitive test which also differentiate between infected and vaccinated animals

Specifically for bTB, the government strategy will also rely on the vaccination of badgers because although badgers (and other feral animals eg deer, boar, etc and dogs do get infected and act as transmission vectors, the main transmission is between themselves and in the cattle herds themselves. Currently there is no practical solution for implementing a vaccination programme for badgers because if they are trapped they need to be tested before determining infected or not (which takes 3 days with the standard test) if infected they get culled, if not, vaccinated and released. The apparatus and test method of the current invention resolves this problem.

Furthermore the biomarker signature which has been identified is valid also for human TB.

Typically, all human and animal diseases, as well as any diagnostic tests requiring full traceability, security, integrity, ability to be interrogated and required for bioinformatics e.g. microbial tests for transmittable diseases, biometric fingerprinting, food chain traceability and control benefit from the invention.

Typically the portion of the apparatus which comes into contact with the sample, such as a lateral flow test portion may be specifically designed for the particular purpose in terms of the type of disease to be tested and/or the risk category of that disease to the person performing the test so as to provide the appropriate masking and protection for them and/or the “type” of person who will be performing the test. i.e. whether they are a health professional, such as a doctor vet, or a non-professional, such as a farmer, carer or the like and with the aim being to enable the test to be performed efficiently and reliably, regardless of who performs it and thereby minimise the risk to the person performing the test. Typically, in whichever form, the body or at least the portion of the body which contacts with the sample will be a single use disposable component.

Typically the apparatus for use on site will be provided to be a hand held body and in addition to the said sample test portion may include any/or any combination of means to receive manually input alphanumerical data, scan barcodes, capture photographic images, perform image recognition, read the test result, possibly store the test result at least on a temporary basis, transmit the data to a remote location such as a cloud database and/or receive data from the subsequent analysis which is performed and will typically have a display screen to allow user interaction with the same.

Thus, in accordance with the invention, there is provided a biochemical biomarker signature for each individual diagnostic test and the individual biomarkers (analytes) and their antibodies. The diagnostic test system algorithms which calculate the outcome of the biomarker assay and report the result will typically derive statistically from validated data to determine the sensitivity and specificity of the assay.

Typically therefore the current invention allows the identification of biomarker signatures relating to the animal or human on which the test has been performed and which are linked to a specific disease in relation to which the test is performed and also in relation to a particular stage of the disease.

This also allows intelligent learning to be performed so that the associated data and metadata can be used to build a knowledge base for where the disease is prevalent, whether there are trends related to location, farm practice, transportation, lifestyles, environments or any combination of these and regardless of whether the apparatus and method is used in relation to animals or humans.

1. Test apparatus for use in testing for presence of at least one health condition with regard to an animal or person on which the apparatus is used, said test apparatus comprising:
a body including a means for identifying the person or animal, a portion with which a sample of tissue and/or fluid from the person or animal is brought into contact and wherein the body includes one or more analysis means, a type of which is selected with respect to a particular test which is to be performed and, after the test is performed, an identification is provided on the body and/or wirelessly transmitted to a remote location to represent results of the test.

2. Apparatus according to claim 1 wherein the one or more analysis means allows analysis of biomarker antibodies and/or antigens.

3. Apparatus according to claim 2 wherein antigens are selected to be used for tests of a type in which an antibody response is not possible.

4. Apparatus according to claim 1 wherein the test apparatus includes a means to allow an indication of a result of the test to be captured and then transmitted and/or stored.

5. Apparatus according to claim 4 wherein the capture is in the form of a photograph or scan.

6. Apparatus according to claim 4 wherein the said indications of test results are communicated to a database to allow the indications to be analysed and, if appropriate, the test result confirmed and a recommended action or range of actions which are to be performed with regard to that particular person or animal or group of persons or animals.

7. (canceled)

8. Apparatus according to claim 6 wherein the action or range of actions which are required to be performed, if required, are communicated to the person or persons who have undertaken the tests on site so as to enable the recommended action or actions to be performed immediately.

9. Apparatus according to claim 1 wherein the one or more analysis means includes a series of biomarker antibodies in a form so as to identify a presence, or otherwise of any of bovine or human tuberculosis, lung diseases including COPD and lung cancer, mycrobacterial disease, such as Johne's disease (mycobacteria avium paratubercolosis (MAP)) prostate and/or bladder cancer.

10. Apparatus according to claim 1 wherein the body portion which contacts with the sample is formed so as to allow a lateral flow test to be performed on the apparatus.

11. Apparatus according to claim 1 wherein the test apparatus includes a further point of use analytical apparatus with a different test and analysis mechanism and so allow the apparatus to include or be adapted to provide a range of differing on site analysis options.

12. (canceled)

13. Apparatus according to claim 11 wherein the said on site analysis options are provided on the body portion and are available to be selectively used.

14. Apparatus according to claim 11 wherein at least the part of the body which includes the portion for contact with the sample is releasable from the remainder of the body and thereby allows a part with an appropriate analysis option for the test which is to be performed, to be fitted to the remainder of the body and thereby adapt the apparatus.

15. Apparatus according to claim 1 wherein the indication generated by the apparatus is a visual indication means.

16. Apparatus according to claim 15 wherein a first colour equates to a clear test result and a second or number of further colours equate to a particular health problem and/or type of health problem.

17. Apparatus according to claim 1 wherein the analysis means include a number of biomarkers, with the number, type and/or specific configuration of the biomarkers selected to suit the particular test which is to be performed.

18. Apparatus according to claim 17 wherein the biomarkers which are detected, rely on any or any combination of contact with the sample from a person or animal in the form of saliva, urine and/or blood.

19. Apparatus according to claim 1 wherein the analysis of the data received from the apparatus uses “omics” research.

20. A method of identifying and providing, if necessary, one of a range of actions with regard to one or more health conditions of a person or animal, said method comprising the steps of; performing a test on a sample obtained from said person or animal to identify results relating to their said health condition; analysing the test results using one or more analysis systems suitable for the test performed and providing an indication of the test result at the location at which the test is performed and/or transmitting data relating to the test results to a remote location for subsequent analysis.

21. A method according to claim 20 wherein the analysis utilises biomarker antibodies and/or antigens.

22. A method according to claim 20 wherein the test utilises a lateral flow system.

23. A method according to claim 20 wherein if the test results indicate a health problem one or more actions are recommended to the person on which the test has been performed and/or person performing the test while they are at the location at which test is performed.

24. A method according to claim 20 wherein the method includes the steps of comparing the data received from the test to a validated database control to determine the status of the tested health condition, reporting the status of the condition to the user and/or tester and uploading the data and the status determined by the test to at least one database in conjunction with a user identification for the test data; and/or storing the test and identification data in a secure manner; and/or interrogating said data using one or more algorithms to compute a decision process regarding the condition; and/or identifying one or more actions which may be required to be performed as a result of the detected condition status and wherein the test is performed using apparatus which identifies one or more biomarkers identified in a bodily fluid from the person or said animal.
